(19) United States
(12) Patent Application Publication
(10) Pub. No.: US 2024/0091367 A1
(43) Pub. Date: Mar. 21, 2024

(54) ORALLY DISINTEGRATING PALATABLE FORMULATIONS OF DROTAVERINE AND METHOD OF PREPARATION THEREOF

(71) Applicant: Sushma Paul BERLIA, New Delhi (IN)

(72) Inventors: Sushma Paul BERLIA, New Delhi (IN); Nishant BERLIA, New Delhi (IN); Aditya BERLIA, New Delhi (IN); Gurvinder SINGH, New Delhi (IN)

(21) Appl. No.: 18/361,142

(22) Filed: Jul. 28, 2023

(30) Foreign Application Priority Data
Jul. 29, 2022 (IN) 202211043456

(51) Int. Cl. A61K 47/58; A61K 9/00; A61K 9/20

(52) U.S. Cl. CPC A61K 47/585; A61K 9/0056; A61K 9/2027; A61K 9/2095

(57) ABSTRACT

The present disclosure provides palatable orally disintegrating tablet formulations of drotaverine or a salt thereof, methods of their preparation, and their use in treatment.

### FIELD

The present disclosure relates to oral palatable formulations of Drotaverine or salt thereof. The present disclosure also relates to oral palatable formulations of Drotaverine or salt thereof in the form of orally disintegrating tablets (ODT), mouth dissolving tablets, dispersible granules, dispersible tablets, or the like. The disclosure also relates to methods for preparation of such formulations along with uses thereof.

### BACKGROUND

Oral administration of drugs is preferred in patients for being non-invasive and having low risk of pain, which leads to improved patient compliance. However, patient groups such as the elderly, children, the intellectually disabled, non-cooperative patients, nauseated patients, patients on reduced liquid-intake or diets, patients with no immediate access to water, patients suffering from Parkinson's diseases, Alzheimer's diseases, dysphagia, post-surgical patients facing difficulty to sit up or patients who are restricted to access to water prior to surgery have difficulty swallowing oral dosage forms. In some cases, like motion sickness, sudden episodes of allergic attack, or coughing, swallowing conventional tablets is difficult. Difficulty in swallowing or dysphagia is seen to afflict nearly 35% of general population. Therefore, orally disintegrating tablets (ODTs) are highly desirable in medical care where the dosage form containing active pharmaceutical ingredients can disintegrate rapidly, usually in a matter of seconds, without the need for water, providing optimal convenience to the patients.

ODTs are also known as mouth dissolving tablets (MDTs), orodispersible tablets, fast disintegrating tablets, quick disintegrating tablets, fast dissolving tablets, rapid dissolving tablets, porous tablets, quick melt tablets and rapid melt tablets. While these terms can be used interchangeably, the approved term for this dosage form by the United States Pharmacopoeia (USP) is orally disintegrating tablets or ODTs (Guidance for Industry: Orally Disintegrating Tablets, 2008). In some embodiments, ODTs offer advantages of solid and liquid dosage forms together with additional non-limiting special advantages as follows:
Accurate dosing: As ODTs are unit solid dosage forms, they provide advantages of accurate dosing, easy manufacturing, small packaging size, and easy handling by the patients.
Rapid action: The rapid disintegration of the ODTs results in quick dissolution of drug and fast absorption that provides a rapid onset of action.
Enhanced bioavailability: An ODT increases bioavailability of an active agent due to absorption of drugs in oral cavity from saliva that pass down into stomach thereby resulting in better efficacy and improved patient compliance.
Ease of administration: ODT formulations are suitable for pediatric, geriatric, and inpatients, especially for the intellectually disabled, psychiatric patients, disabled and bedridden patients or patients who do not have access to water, patients who are traveling, etc.
ODTs have the advantages of solid dosage form, especially ease of handling, and liquid dosage forms including ease of swallowing and pre-gastric absorption.
Obstruction free: ODTs do not suffer from risk of suffocation in airways caused by physical obstruction when swallowed, thereby providing improved safety and compliance.

One of the major limitations in formulating ODTs is the lack of mechanical strength of the tablets. ODTs have a porous and soft molded matrix, which is then compressed in a tablet form using low compression, creating a friable and brittle tablet that is difficult to handle.

An ideal ODT should exhibit rapidly disintegrating properties in the oral cavity and should have sufficient physical strength (i.e., hardness) so it retains its physical integrity during the steps of manufacturing, distribution, and patient handling, such as while taking out the tablets from packaging.

Taste is another important parameter for drugs administered orally. Undesirable tasting or bitter tasting drugs pose a major challenge for formulation scientists for developing palatable orally disintegrating formulations. Additionally, minimal or no residue in the mouth is advantageous for ODTs as any large particles from the disintegrating tablet which are insoluble or slowly soluble in saliva can lead to an unpleasant gritty feeling or lingering unpleasant aftertaste.

Drotaverine HCl is a benzyl isoquinoline derivative and is an analogue of papaverine. Drotaverine is a highly potent antispasmodic agent. Mode of action and pharmacokinetic parameters of Drotaverine along with the diseases, disorders, or physiological conditions for which Drotaverine is used is well known. Drotaverine is known to effectively contribute either alone or in combination in relieving painful spasms in wide variety of diseases and is the drug of choice in emergency medicine. Drotaverine does not have side effects like anticholinergics and it does not mask symptoms of “acute abdomen.”

Abdominal pain is a common complaint in adults and children. Drotaverine is an effective and safe pharmaceutical agent in management of recurrent abdominal pain of children. It also has been used as an off-label medication for alimentary tract problems in pre-school and school-aged children. In treatment of acute conditions, it is desired that pharmaceutical compositions have a rapid and consistent onset of action and good bioavailability. Currently marketed preparations of Drotaverine injection are suitable for such acute conditions as rapid absorption can be achieved by parenteral injection, but this is undesirable to many patients, particularly in emergency situations or while travelling, where health care professionals are not accessible. In such a scenario, ODTs can be a suitable alternative.

In addition to injection, Drotaverine is currently marketed as a conventional immediate release (IR) product such as IR film coated tablets and syrup. Drotaverine is available in strengths of 40 mg and 80 mg tablets and the prescribed dose is 120-240 mg/day. The available Drotaverine IR film coated tablets typically require water for oral administration and hence are not suitable for some patient populations.

Drotaverine is an extremely bitter drug with a lingering aftertaste. While there are approaches that are effective in masking the taste of a bitter drug, it is known that an approach that works with one drug substance often does not apply to another drug substance.

Drotaverine is also highly susceptible to oxidation and pH, leading to chemical instability. A number of techniques have been investigated so far to mask the taste of Drotaverine HCl, such as solid dispersion, drug coatings, and complexation with polymers or resins. However, successful formulation of a chemically stable and palatable ODT formulation of Drotaverine has not been reported in literature. Therefore, there is a need to treat patients with inability or unwillingness to swallow currently available Drotaverine formulations.

Currently available techniques for making ODT formulations include use of lyophilization, cotton candy, sublimation, melt extrusion, and direct compression, in addition to conventional wet granulation processes. Among different manufacturing processes, direct compression is one of the most economical methods, as it uses conventional equipment, commercially available excipients, and relatively simple process steps. However, the disintegration capacity of ODTs produced by direct compression typically is limited by size and hardness of the resulting tablets. Therefore, developing a dosage form with high disintegration capacity typically requires the use of excipients that offer both cohesiveness for compaction and subsequent disintegration when the dosage form comes in contact with saliva in the mouth.

US20090136569A1 discloses a rapidly disintegrating tablet in an oral cavity, which contains three components: 1) a granule containing Bepotastine Besilate in which the bitter taste of the drug has not been suppressed; 2) a granule containing a water-soluble saccharide and a binder; 3) excipients including a flavoring ingredient, a sweetener, and a lubricant. However, the process of manufacturing the tablet is lengthy, has poor production efficiency, and suffers from reduced content uniformity due to granule classification.

US20060172005A1 discloses a tablet that rapidly disintegrates in oral cavity wherein the tablet comprises an active ingredient and 70% by mass or more of cyclodextrin or a derivative thereof. However, such tablet suffers from poor texture because of large amount of cyclodextrins. Further, cyclodextrin has high hygroscopicity which can affect hardness of the tablet during storage.

US20090311321A1 discloses a bitterness-masked ODT of mitiglinide calcium comprising microcrystalline cellulose, at least one masking agent, a sugar or a sugar alcohol, and at least one member selected from corn starch and partially pregelatinized starch.

U.S. Pat. No. 7,510,728B2 discloses a quickly disintegrating solid pharmaceutical preparation of pioglitazone hydrochloride.

EP2808013A1 discloses an ODT produced by compressing a mixture of avanafil, nicergoline, imidapril hydrochloride, bisoprolol fumarate, taltirelin hydrate, and allopurinol.

Despite the efforts made to date, none of the known art has been able to overcome the disadvantages of Drotaverine to provide a chemically stable and palatable oral formulation suitable for ODT. Hence, there is a need to develop a chemically stable and palatable ODT of Drotaverine or salt thereof for high acceptability, faster onset of action for early pain relief, and improved convenience for people who have difficulty in swallowing. Further, such formulations should be easily prepared by processes such as direct compression, dry granulation, or wet granulation processes. Additionally, in some embodiments, size of the ODTs should be as small as possible to leave minimal or no residue after disintegration in the mouth cavity. In some embodiments, the tablet should disintegrate in the mouth cavity, preferably in less than 30 seconds, when it comes in touch with saliva as no water will be used for swallowing the ODTs.

### SUMMARY

The present disclosure is related Orally disintegrating tablets (ODTs) comprising Drotaverine or a pharmaceutical salt thereof, that disintegrate in saliva and are swallowed without the need for water. These disclosed formulations offer a significant advantage over the currently marketed conventional film coated swallowing tablets especially for geriatric patients who usually face difficulty in swallowing such tablets. The disclosed pharmaceutical compositions are chemically stable throughout the product's shelf-life. Using a novel manufacturing process, the disclosed formulations exhibit improved taste, improved aftertaste, and improved palatability.

In some embodiments, the present disclosure provides for an oral dosage form comprising Drotaverine or a salt thereof and an ion-exchange resin, wherein Drotaverine and the ion-exchange resin comprise a Drotaverine-resin-exchange complex, and wherein the dosage form is formulated as an orally disintegrating tablet. In some embodiments, the present disclosure provides for an oral dosage form comprising Drotaverine or a salt thereof and a taste enhancer. In some embodiments, the dosage form comprises a bitter masking flavor. In some embodiments, the dosage form comprises 2.5-100 mg Drotaverine or a salt thereof. In some embodiments, the dosage form comprises 10-100 mg Drotaverine or a salt thereof. In some embodiments, the dosage form comprises Drotaverine hydrochloride. In some embodiments, the ion-exchange resin is Kyron 114, Kyron 314, Indion 204, Indion 234, Indion 294, or a combination thereof. In some embodiments, the ratio of Drotaverine to ion-exchange resin is 1:2 to 1:6. In some embodiments, the oral dosage form is in the form of granules, wherein the size of the granules is in the range of around 850 microns to 300 microns. In some embodiments, the oral dosage form of any of claims 1-4, wherein the dosage form has a hardness of 3-12 kp. In some embodiments, the dosage form disintegrates in the mouth in 60 seconds or less without water. In some embodiments, the dosage form disintegrates in the mouth in 30 seconds or less without water. In some embodiments, the dosage form disintegrates in 1-3 mL of water in 60 seconds or less. In some embodiments, the dosage form disintegrates in 1-3 mL of water in 30 seconds or less.

In some embodiments, the dosage form further comprises a diluent, a disintegrant, a flavoring agent, a sweetener, a lubricant, a glidant, an anti-adherent, or a combination thereof. In some embodiments, the ratio of excipients to drug-resin complex is 0.05:1 to 2.5:1. In some embodiments, the ratio of excipients to drug-resin complex is 0.1:1 to 2:1.

In some embodiments, the dosage form further comprises at least one diluent, wherein the diluent comprises a monosaccharide, an oligosaccharide, a polysaccharide, a lactose, a sugar alcohol, a cellulose powder, a microcrystalline cellulose, a silicified microcrystalline cellulose, a derivative of cellulose modified chemically, a starch, or a combination thereof.

In some embodiments, the dosage form further comprises at least one disintegrant, wherein the disintegrant comprises at least one of a starch, natural or chemically modified cellulose, microcrystalline cellulose, gum, alginic acid or salt thereof, sugar, aluminum oxide, or a synthetic polymer. In some embodiments, the disintegrant is in an amount of 1% to 10% w/w. In some embodiments, the disintegrant is in an amount of 2% to 8% w/w.

In some embodiments, the dosage form further comprises at least one flavoring agent, wherein the flavoring agent comprises a strawberry flavor, a cherry flavor, an orange flavor, a peppermint flavor, a black currant flavor, a banana flavor, a raspberry flavor, a red fruit flavor, a wild berry flavor, a caramel flavor, or a combination thereof.

In some embodiments, the dosage form further comprises at least one sweetener, wherein the sweetener comprises aspartame, potassium acesulfame, sodium saccharinate, neohesperidine dihydrochalcone, sucralose, sucrose, fructose, monoammonium glycyrrhizinate, or a combination thereof.

In some embodiments, the dosage form further comprises at least one lubricant, wherein the lubricant comprises sodium benzoate, sodium stearyl fumarate, calcium stearate, magnesium stearate, zinc stearate, glyceryl behenate, stearic acid, glyceryl monostearate, or a combination thereof.

In some embodiments, the present disclosure provides for an oral dosage form comprising Drotaverine or a salt thereof and an ion-exchange resin, wherein the oral dosage form is stable for three months when stored at 25-30° C. and 60-75% relative humidity (RH) as long-term storage conditions. In some embodiments, the dosage form is stable for six months when stored at 25-30° C. and 60-75% RH as long-term storage conditions. In some embodiments, the dosage form is stable for two years when stored at 25-30° C. and 60-75% RH as long-term storage conditions. In some embodiments, the dosage form is stable for three months when stored at 40° C. and 75% RH as accelerated storage conditions. In some embodiments, the dosage form is stable for six months when stored at 40° C. and 75% RH as accelerated storage conditions. In some embodiments, the oral dosage form exhibits improved taste. In some embodiments, the oral dosage form exhibits improved aftertaste. In some embodiments, the oral dosage form exhibits improved palatability.

In some embodiments, the present disclosure provides for a method of preparing an orally disintegrating tablet of Drotaverine or salt thereof comprising: (a) mixing an ion-exchange resin with water; (b) adding Drotaverine or salt thereof to the ion-exchange resin mixture to form a drug-resin complex; (c) drying the drug-resin complex to form a dried drug-resin complex; (d) sieving the dried drug-resin complex to obtain drug-resin complex granules; (e) separately obtaining and weighing the drug-resin complex granules and at least one excipient; (f) passing the drug-resin complex granules and excipient through a mesh; (g) blending the drug-resin complex granules and excipient; and (h) forming the blended drug-resin complex granules and excipient into an orally disintegrating tablet. In some embodiments, the ion-exchange resin comprises Kyron 114, Kyron 314, Indion 204, Indion 234, Indion 294. In some embodiments, the ion-exchange resin is mixed with water for 30-45 minutes, then Drotaverine is added slowly and mixed for at least 2-4 hours. In some embodiments, the ion-exchange resin and Drotaverine or salt thereof are mixed in a ratio of 2:1 to 6:1. In some embodiments, the drug-resin complex is air dried or dried using drying equipment. In some embodiments, the dried drug-resin complex has less than 5.0% w/w of water. In some embodiments, the dried drug-resin complex is sieved through a mesh, wherein the mesh is no larger than 850 microns. In some embodiments, the drug-resin complex granules and excipient are mixed for 10-20 minutes. In some embodiments, the orally disintegrating tablet is formed using direct compression, dry granulation, wet granulation, or fluidized bed preparation methods. In some embodiments, at least 90% of the orally disintegrating tablet is dissolved in five minutes in vitro using a paddle at 40 RPM in 0.1 N HCl dissolution media.

In some embodiments, the present disclosure provides for a method of treating abdominal pain comprising administering to a subject in need thereof an oral dosage form comprising Drotaverine or a salt thereof, wherein the oral dosage form comprises any of claims 1-24.

In some embodiments, the present disclosure provides for use of an oral dosage form comprising Drotaverine or a salt thereof to treat abdominal pain comprising administering the oral dosage form to a subject in need thereof, wherein the oral dosage form comprises any of claims 1-24.

### DETAILED DESCRIPTION

The bitterness of pharmaceutical medicines is a consideration in patient compliance, as the oral administration of bitter drugs can be hampered by their unpleasant taste which leads to non-compliance or patient's inability to swallow the drug, thereby worsening the diseased condition. The present disclosure is related to ODTs comprising Drotaverine or a pharmaceutical salt thereof.

In some embodiments, the present disclosure provides palatable ODT formulations of an antispasmodic agent, particularly Drotaverine or salt thereof, that disintegrate quickly in the oral cavity, particularly in less than 30 seconds.

In some embodiments, the present disclosure provides ODT formulations of multiple strengths of Drotaverine or salt thereof. In some embodiments, these formulations provide flexible dose ranges suitable for different groups of patients. In some embodiments, the present disclosure provides methods for preparing the palatable ODT formulations of Drotaverine or salt thereof. In some embodiments, the present disclosure provides uses of the oral formulations.

In some embodiments, the ODT formulations of the present disclosure have sufficient mechanical resistance to withstand crumbling or breaking during production and distribution operations, unlike other fast disintegration formulas such as oral lyophilizates, tablets of saccharide-based shear form floss, and wafers.

In some embodiments, the ODT formulations of the present disclosure exhibit chemical stability throughout long-term storage conditions.

In some embodiments, the disclosure provides ODTs of Drotaverine that exhibit improved taste, aftertaste, and palatability. The term “taste” is used herein to refer to the sensation of flavor experienced when a subject's tongue comes into contact with a dosage form. The term “aftertaste” is used herein to refer to the sensation of flavor experienced in the mouth after administration of a dosage form. For example, an aftertaste typically lasts well after administration of Drotaverine. The term is used herein to refer to the physical sensation of a dosage form when it is in a subject's mouth, such as the texture of a dosage form. In some embodiments, the disclosed ODTs disclosed herein exhibit improved taste, aftertaste, and palatability compared to presently available Drotaverine tablets.

The terms “stable,” “stability,” or “stabilized” mean the dosage form is able to maintain a product performance test parameters such as an ingredient amount, a related substance amount, without significant changes from the initial levels.

The term “significant change” means a change of 5% or more from the initial result. For example, if the initial amount of an ingredient is 98%, then a significant change would be an amount of 93% or less of that ingredient. If the product is stable under accelerated conditions (40° C./75% RH) for 6 months without significant change, then it can be assigned a shelf life of at least 2 years or 24 months. In some embodiments, the ODT is stable under long-term storage conditions for three months. In some embodiments, the ODT is stable under long-term storage conditions for six months. In some embodiments, long-term storage conditions comprise 15-30° C. at 60-75% RH. In some embodiments, long-term storage conditions comprise 25-30° C. at 60-75% RH.

In some embodiments, the ODT formulations disclosed in present disclosure are suitable for all age groups, including but not limited to the elderly, children, the intellectually disabled, non-cooperative patients, nauseated patients, patients on reduced liquid-intake or diets, patients with no immediate access to water, patients suffering from Parkinson's diseases, Alzheimer's diseases, dysphagia, post-surgical patients facing difficulty to sit up or patients who are restricted to access water prior to surgery have difficulty swallowing oral dosage forms.

In some embodiments, the ODT formulations of the present disclosure mask the bitter taste of Drotaverine. During development of the palatable ODT formulations of the present disclosure, various methods and combination approaches were tried to overcome sensory challenge of bitter taste and lingering after taste of the Drotaverine, including the ion-exchange resins approach.

While the ion-exchange resin approach masked the strong unpleasant bitterness with a lingering after-taste of Drotaverine, the resulting product did not provide acceptable palatable characteristics. For example, the resulting product exhibited grittiness and included bulky residue after administration. To address these issues, additional experiments were conducted. This led to development of ODT formulations with appropriate granule size and suitable ratio of drug-resin complex to achieve the desired texture. Finally, a combination of ion-exchange resins with blends of excipients and flavors resulted in a palatable ODT formulation of Drotaverine. The developed ODT formulations of the present disclosure have minimal excipients, which helps keep tablet size small and reduces the amount of residue in the mouth after disintegration. This results in the ODTs of present disclosure having a pleasant mouth feel.

Though ion-exchange resin technology can be employed for taste masking, the developed formulations surprisingly also exhibited improved chemical stability.

In some embodiments, the ODTs of the present disclosure disintegrate rapidly in less than 30 seconds in the oral cavity without water. In some embodiments, the developed ODTs provide content uniformity and strength of compressed tablets, ensuring delivery of intact tablets to patients up until administration. In some embodiments, the ODT formulations of present disclosure provide quick release of Drotaverine, providing prompt pain relief to the patients. In certain embodiments, ODT formulations of the present disclosure have no adverse effect on drug dissolution release despite use of ion-exchange resin complexation technology. In some embodiments, ODT formulations of the present disclosure are prepared by a cost effective and easily scalable manufacturing process.

In some embodiments, ODT formulations of the present disclosure contain ingredients commonly used in drug industry, herein referred to as conventional excipients but not limited to diluents, binders, disintegrants, flavouring agents, sweeteners, lubricants, glidants, anti-adherents, and mixtures thereof.

Examples of diluents include saccharides such as monosaccharides, oligosaccharides or polysaccharides, lactose in its various forms, anhydrous, monohydrate, sugar alcohols such as mannitol, maltol, sorbitol, maltitol, xylitol, isomalt and erythritol, cellulose powder, microcrystalline cellulose, silicified microcrystalline cellulose or derivatives of cellulose modified chemically, such as hydroxypropyl cellulose, hydroxypropyl methyl cellulose, starch of different forms e.g. maize, corn, potato starches, sucrose, or mixtures thereof.

The term “disintegrant,” used herein refers to a compound which facilitates break-up or disintegration of a tablet when placed in aqueous environment. Disintegrants swell when they come in contact with water, changing in volume or form that produces a disruptive force that causes a compressed tablet to break apart. Examples of disintegrants include natural starches, such as maize starch and potato starch, directly compressible starches such as starch 1500, modified or pregelatinized starches such as carboxymethyl starches and sodium starch glycolate; natural or chemically-modified cellulose, especially crosslinked sodium carboxymethyl cellulose (croscarmellose sodium) or low substituted hydroxypropyl cellulose; microcrystalline cellulose, gum, especially agar gum, and guar gum; alginic acid or salts thereof acetates and citrates, sugars (especially lactose, mannitol and sorbitol), aluminum oxide, and synthetic polymers such as crospovidone. In some embodiments, the disintegrant is in an amount of 1% to 10% w/w. In some embodiments, the disintegrant is in an amount of 2% to 8% w/w.

In some embodiments, ODT formulations of the present invention comprise a binder. In some embodiments, the binder is a starch, polyvinylpyrrolidone, a natural gum, a synthetic gum, a cellulosic polymer, or a combination thereof.

In some embodiments, ODT formulations of the present invention comprise a lubricant, a glidant, or a combination thereof. In some embodiments, the lubricant and glidant is talc, colloidal silicon dioxide, stearic acid, sodium stearyl fumarate, magnesium stearate, or a combination thereof.

Examples of the suitable flavoring agents used in formulations of the present disclosure include lemon flavor, anise flavor, cinnamon flavor, chocolate powder flavor, strawberry flavor, cherry flavor, orange flavor, peppermint flavor, black currant flavor, banana flavor, raspberry flavor, red fruits flavor, wild berries flavor, and caramel flavor. In some embodiments, the flavoring agents are used in an amount of less than 3.0% by weight.

Certain flavoring agents are especially effective at masking the bitter aftertaste of Drotaverine. These flavoring agents are referred to as bitter masking flavors. In some embodiments, the formulations of the present disclosure comprise a bitter masking flavor. In some embodiments, the bitter masking flavor comprises peppermint flavor, lemon flavor, anise flavor, cinnamon flavor, chocolate powder flavor, or combinations thereof.

Examples of the suitable sweeteners used in formulations of the present disclosure include aspartame, potassium acesulfame, sodium saccharinate, neohesperidine dihydrochalcone, sucralose, sucrose, fructose, monoammonium glycyrrhizinate, licorice powder, cinnamon powder, or a combination thereof. In some embodiments, the sweetener is used in an amount of about 1.0 to 4.0% by weight.

Certain sweeteners are especially effective at masking the bitter aftertaste of Drotaverine. These sweeteners are referred to as taste enhancers. In some embodiments, the formulations of the present disclosure comprise a taste enhancer. In some embodiments, the taste enhancer comprises licorice powder, cinnamon powder, monoammonium glycyrrhizinate (MAG), or a combination thereof.

In some embodiments, lubricants used herein are additional excipients that can affect performance of an oral-dispersible pharmaceutical formulations. Suitable examples of lubricants include sodium benzoate, sodium stearyl fumarate, calcium stearate, magnesium stearate, zinc stearate, glyceryl behenate, stearic acid, and glyceryl monostearate. In some embodiments, lubricant for formulation of the present disclosure is sodium stearyl fumarate or magnesium stearate or combination thereof. In some embodiments, the lubricants are used in an amount of about 0.25 to 5% by weight.

The development of the ODTs of the present disclosure is briefly described below.

During the initial studies of development of present disclosure, several experiments were performed with combinations of diluents, sweeteners, flavoring agents, and Drotaverine HCl. The manufacturing processes tried were direct compression, dry granulation, wet granulation using high shear granulation, and fluidized bed process methods. Even though some formulations were able to achieve rapid disintegration of tablets during initial development, the final product did not provide desired mouth feel. In addition, high impurity levels observed in the final tablet were beyond the limits recommended by the International Council for Harmonization of Technical Requirements for Pharmaceuticals for Human Use (ICH) guidelines. Additionally, bitterness due to Drotaverine could not be masked with these conventional methods and use of combination of flavoring and sweetening agents.

In order to mask the bitterness of Drotaverine, the ion-exchange resins approach was tried. Ion-exchange resins are solid and suitably insoluble high molecular weight polyelectrolytes that can exchange their mobile ions of equal charge with the surrounding medium. An ion-exchange resin is an insoluble matrix (or support structure) normally in form of small (1-2 mm diameter) beads, usually white or yellowish, fabricated from an organic polymer substrate. The material has highly developed structure of pores on the surface which are sites with easily trapped and released ions. Trapping of ions takes place with simultaneous releasing of other ions; thus, the process is called ion-exchange. There are multiple different types of ion-exchange resin which are fabricated to selectively prefer one or several different types of ions. In some embodiments, suitable ion exchange resins used herein are cross linked polyacrylic acid polymers and their salts such as Kyron grades viz. Kyron 114, Kyron 314 and Indion range of resin grades viz. Indion 204, Indion 234 & Indion 294.

Ion-exchange resins are known as polymeric materials which have the possibility to form weak bonds with drugs having positive charge. They are insoluble polymers which contain acidic or basic functional groups and have the ability to exchange counter ions with aqueous solution surrounding them. Complexation of the drug and ion-exchange resin is based on the drug loading ratio (ratio of active ingredient and resin), temperature, and pH. In some embodiments, the active ingredient is loaded onto an ion-exchange resin by dissolving or dispersing an acidic or basic, ionizable active ingredient in water, and then mixing it with a suitable ion-exchange resin.

A representation of the ion-exchange process showing acid-base complexation formed from a positively charged drug (D+) and an ion-exchange resin (RCOO−) is depicted below:

D⁺+RCOO⁻→RCOO-D (formulation in mouth)

RCOO-D+H⁺+Cl⁻→D+RCOOH+Cl⁻ (formulation is stomach)

Ion-exchange resins mask the taste of a drug because the resin-drug complexes that form only elute little to no drug in near neutral pH of the mouth. Thus, taste of the drug is masked in the mouth without interrupting the drug release profile in the stomach.

Several experiments were conducted in order to develop suitable Drotaverine drug-resin complex, which results in masking of bitterness and lingering after-taste. The drug and suitable grade of ion-exchange resins were selected based on initial studies, wherein, drug to polymer ratio employed was ranging from 1:1 to 1:8.

Ion-exchange resins were initially mixed in water and stirred for sufficient time under suitable temperature and pH. Thereafter, Drotaverine was added slowly into resin mixture and stirred for sufficient time to obtain resinate or drug-resin complex. The resinate obtained was separated by filtration using suitable means. Thereafter, the filtered drug-resin complex was washed until filtrate obtained after washing was nearly free of unbound or free Drotaverine drug. The free Drotaverine drug unbound to resin should not exceed 2% w/w of the input drug at the start of complexation process; preferably it should be less than 1% w/w. Thereafter, the drug-resin complex is air dried overnight at room temperature or dried using suitable drying equipment under suitable temperature conditions till desired water content is achieved. The suitable drying equipment for drying of drug-resin complex include, but not limited to, tray dryer or fluidized bed dryer until target water content of less than 5.0% w/w is achieved. The dried drug-resin complex is tested for free Drotaverine drug. The free drug content should not be more than 2.0%.

The dried drug-resin complex was thereafter used for formulation of oral formulations by using suitable pharmaceutical excipients or additives which may include sweetening agents, flavors, colorants, antioxidants, chelating agents, surfactants, wetting agents, antifoaming agents, pH modifiers, acidifiers, preservatives, co-solvents, and mixtures thereof.

Based on extensive experiments with different combinations, the drug-resin complex in the ratio of 1:2 to 1:6 was finalized and after drug-resin complexation and drying step, granule size of the drug-resin complex was finalized. In order to ensure smooth texture and palatability aspects, the dried drug-resin complex granules particle size was sufficiently reduced with appropriate milling parameters to ensure that the final product is free of grittiness and provide smooth feel while swallowing.

Based on the above extensive studies, the appropriate granule size was established which is not more than 20 mesh, ASTM (equivalent to around 850 microns), preferably 30 mesh, ASTM (equivalent to around 600 microns) or more preferably around 50 mesh, ASTM (equivalent to around 300 microns).

Once the desired particle size specifications of the drug-resin complex were firmed up, further formulation experiments were conducted for mouth dissolving tablet formulation. Though, bitterness of Drotaverine drug was masked using suitable ion-exchange resin in desired drug-resin ratios, it was not sufficient to provide the desired palatability of orally disintegrating mouth dissolving tablets. In order to get desired pharmaceutical performance parameters along with the acceptable mouth feel, further development studies were conducted to achieve appropriate ratios of ‘pharmaceutical excipients’ in addition to using drug-resin complexes as core materials Therefore, additional ingredients herein referred to as ‘pharmaceutical excipients’ or ‘additives’ were used for formulating the final mouth dissolving tablet product. The pharmaceutical excipient may be selected from diluents, binders, lubricants, disintegrants, coloring agents, stabilizers, glidants, etc. In some embodiments, the ratio of pharmaceutical excipients to the drug-resin complex is from 0.05:1 to 2.5:1. In some embodiments, the ratio of pharmaceutical excipients to the drug-resin complex is from 0.1:1 to 2:1.

Additional additives may include taste enhancers, bitter masking flavors, antioxidants, chelating agents, surfactants, wetting agents, antifoaming agents, pH modifiers, acidifiers, preservatives, and mixtures thereof.

In some embodiments, ODT formulations of the present invention comprise an acidifying agent, an acidifier, a pH modifier, or a combination thereof. In some embodiments, the acidifying agent, acidifier, or pH modifiers is citric acid, fumaric acid, lactic acid, maleic acid, malic acid tartaric acid, or a combination thereof.

In some embodiments, the ODT formulations comprise an antioxidant. In some embodiments, the antioxidant is butylated hydroxyanisole (BHA), butylated hydroxytoluene (BHT), sodium metabisulfite, sodium thiosulfate, propyl gallate, ascorbic acid, glycine, cysteine, or combinations thereof.

In some embodiments, the ODT formulations comprise a wetting agent. In some embodiments, the wetting agent is a long alkyl chain sulfonate, long alkyl chain sulfate, quaternary ammonium salt, fatty alcohol, fatty acid ester, polyoxyethylene derivative of a fatty acid ester, or combinations thereof. In some embodiments, the long alkyl chain sulfate is sodium dodecylbenzene sulfonate, sodium lauryl sulfate, dialkyl sodium sulfosuccinate, or a combination thereof. In some embodiments, the fatty alcohol is a laurylester, cetylester, sterylester, glycerylester, or combination thereof. In some embodiments, the polyoxyethylene derivative is synthesized by the addition of an ethylene oxide via polymerization to obtain sorbitan fatty acid esters. These are non-ionic hydrophilic surfactants commonly known as the Tweens or Polysorbates, e.g., polysorbate 20, 60, and 80.

In some embodiments, the ODT comprises a preservative. In some embodiments, the preservative is a parabens or sodium salt thereof. In some embodiments, the parabens is a methyl parabens, propyl parabens, or sodium salts thereof. In some embodiments, the preservative is a benzyl alcohol Thiomersal, or combination thereof.

The ODTs of present disclosure can be prepared by direct compression, dry granulation, or wet granulation processes. The term “direct compression” is used in the context of present disclosure to define a process by which tablets are compressed directly from powder blends of the drug-resin dried complex and excipients, which flow uniformly into a die cavity and form a firm compact. The term “dry granulation” process is used in the context of the present disclosure to define a process by which a final drug product is produced by the steps of preparing flowable blend of granules of precise particle size distribution using slugging or roller compaction process, followed by sizing of slugs or compacts and subsequent lubrication before tablets are compressed. The term “wet granulation” process involves size enlargement in which fine powder particles are agglomerated or brought together into larger, strong and relatively permanent structure called granules using a suitable non-toxic granulating fluid. The drug-resin complex may be used as such or granulated with at least one pharmaceutical excipient and then used for the preparation of a dosage form.

The compressed tablet weight may range from 125 mg to 500 mg depending on suitable ratio of complexing agent, diluents, disintegrants, lubricants, sweeteners, and flavors, for different strengths of Drotaverine formulations as per present disclosure.

In some embodiments, the compressed ODTs containing coated resin particles disintegrate quickly into individual particles in the oral cavity when they come in contact with saliva. Moreover, in some embodiments, the drug release rate is not affected by the complexation and compaction process involved during the manufacturing process.

In order to achieve pleasant mouthfeel, the inventors also investigated different combinations of diluents and taste enhancing ingredients for achieving desired taste. In some embodiments, the selected bulk diluents can be water soluble or dispersible in combination with less water soluble, swellable excipients in the desired ratios.

Examples of routinely used diluents include microcrystalline cellulose, silicified microcrystalline cellulose, or derivatives of cellulose modified chemically, such as hydroxypropyl cellulose, hydroxypropyl methyl cellulose, starch of different forms, for example, maize, corn, potato starches, sucrose, or mixtures thereof.

The inventors also conducted experiments with water soluble diluents such as saccharides. Examples of saccharides containing diluents include monosaccharides, oligosaccharides or polysaccharides, lactose in its various forms, anhydrous, monohydrate, sugar alcohols such as mannitol, maltol, sorbitol, maltitol, xylitol, isomalt, erythritol, and cellulose powder.

Different grades of sugar alcohols in the form of powder and directly compressible grades of sorbitol, xylitol and mannitol are commercially available and have physical and chemical properties that make them ideal for constituting an appropriate diluent. In some embodiments, the directly compressible (DC) grades of sugar alcohols possess good flowability and compressibility characteristics along with excellent mouthfeel when used in appropriate quantities. Few examples of DC grade sugar alcohols include Prosolv ODT, Ludiflash, Pharmaburst, and F-Melt.

Extensive studies were carried to arrive at appropriate ratio of sugar alcohols and routine diluents such as microcrystalline cellulose (MCC), starch, lactose alone or in combination as a co-processed DC grade materials to achieve suitable formulations having excellent compressibility with disintegration in less than 30 seconds, preferably less than 20 seconds when kept in the mouth cavity. Additionally, these selected excipients have good dilution capacity due to the size and form of the particle, which makes it possible to incorporate large amounts of active ingredients that are not easily compressed and allows keeping the final tablet product size to the minimum.

The flavoring agents used were of type and amount desired to enhance the palatability of the formulation. Flavoring agents that may be used in the present disclosure include natural flavors, natural fruit flavors, artificial flavors, artificial fruit flavors, flavor enhancers, or mixtures thereof. Natural flavors, artificial flavors or mixtures thereof include Licorice, mint (such as peppermint or spearmint) and menthol. Natural fruit flavors, artificial fruit flavors or mixtures thereof include cherry, grape, strawberry, orange, melon, banana, citrus, Tutti-fruity and vanilla. Although flavoring agents are generally provided as a minor component of the taste masking composition in amounts effective to provide a palatable flavor to the pharmaceutical formulation, the addition of at least one flavoring agent may be used, and in some embodiments, more than one flavoring agent may be employed.

In some embodiments, the ODT comprises an effervescent agent, which can be added to improve the mouth feel. In some embodiments, the effervescent agent is sodium bicarbonate, citric acid, or a combination thereof. In some embodiments, the flavoring agent or effervescent agent is used in addition to other taste masking techniques to achieve the target taste profile of the product.

In some embodiments, the ODT comprises a coloring agent. Coloring agents or colorants may be incorporated to provide an appealing color to the pharmaceutical formulation. In some embodiments, the coloring agent is deemed safe for human consumption by relevant governmental regulatory bodies and avoids chemical incompatibilities with other ingredients. In some embodiments, the coloring agent is sunset yellow FCF (known as FD&C yellow No. 6 and Tartrazine in the US) which is a synthetic lemon-yellow azo dye. It is also known as FD&C Yellow 5.

Based on the extensive experiments with different combinations of the above-mentioned diluents along with suitable disintegrants, sweetening agent, flavors, few formulation prototypes were selected and disintegration of resultant tablets were found to be less than a minute, even less than 20 seconds. These developed prototypes were evaluated for chemical stability on accelerated stability conditions. The observed impurity levels in the resultant product were found to comply with limits for impurities as per the ICH guideline.

The drug-resin complexation formulations were evaluated with an objective of taste masking. Surprisingly, however, a significant improvement in the chemical stability of Drotaverine in the drug-resin complex was observed compared to formulations without the ion-exchange resin. Additionally, the developed ODT exhibited quick action as almost 90% drug release was achieved in about 5 minutes.

The drug-resin complex prepared was employed for manufacturing the ODT. In some embodiments, methods of preparation of the ODT of present disclosure comprises steps of:
a. weighing required amount of suitable ingredients as per different formulations;
b. passing the required materials through suitable sieve separately;
c. passing sweetener and extra-granular lubricants through suitable sieve separately;
d. blending drug-resin complex along with required diluents by suitable means for sufficient time;
e. blending product of step-d with suitable lubricant by suitable means for sufficient time.

To measure compression of the ODTs, a die-punch tool was used. The prepared tablets were having hardness of about 3 to 12 kp with disintegration time of less than a minute, preferably 10 to 30 seconds.

In summary, the present disclosure provides the orally disintegrating palatable formulations containing chemically stabilized taste masked Drotaverine which disintegrates in oral cavity when it comes in contact with saliva preferably in less than 30 seconds, without the need of water for oral administration. In some embodiments, the drug release rate of the drug-resin formulations of the present disclosure is faster than currently marketed film coated tablets, which leads to faster action and improved absorption.

As representative suitable ODT formulations consistent with the objects, features and advantages of the present disclosure, the following non-limiting examples are provided.

### Example 1: Method for Preparation of Drug-Resin Complex

Ion-exchange resins were initially mixed in 4 to 8 volumes of water and stirred for about 30 to 45 minutes using a mechanical stirrer. Thereafter, Drotaverine HCl was added slowly into the resin mixture and stirred for at least 2 to 4 hours to obtain the drug-resin complex. The drug-resin complex was prepared by employing drug and resin in ratio of 1:2 to 1:6. The drug-resin complex obtained was separated by filtration using a 30# Sieve (ASTM) to obtain filtered drug-resin complex. In some embodiments, a 50# Sieve can be used. Thereafter, the filtered drug-resin complex was washed with water until the filtrate obtained after washing was nearly free of unbound Drotaverine drug.

The drug-resin complex that was obtained was air dried overnight at room temperature or dried using a fluidized bed dryer at 50-70 degrees Celsius. The drying of drug-resin complex was carried out until a water content of less than 5.0% w/w was achieved. The dried drug-resin complex was tested for unbound Drotaverine drug. The unbound Drotaverine drug content should not be more than 2.0%.

The dried drug-resin complex can either be used immediately for formulation of ODT or can be stored for later use. For formulation of the ODT, the excipients listed in Examples 3-18 below were added to the dried drug-resin complex as described in Example 2 below.

Before mixing the dried drug-resin complex with the excipients, the granule particle size for the dried drug-resin complex was reduced using a milling machine. A multi-mill or oscillating granulator milling machine can be used. The appropriate granule size is less than 20 mesh, American Standard Test Sieve Series (ASTM) (equivalent to around 850 microns), 30 mesh, ASTM (equivalent to around 600 microns) or 50 mesh, ASTM (equivalent to around 300 microns).

Once the desired particle size specifications of drug-resin complex was determined, further formulation experiments were conducted for preparing mouth dissolving tablets as described in Example 2.

### Example 2: Method for Preparation of Mouth Dissolving Tablets from Drug-Resin Complexes

The drug-resin complex prepared in Example 1 was employed for manufacturing ODTs. The process comprised the following steps:
a. separately weighing the drug-resin granules and excipients described in Examples 3-18 below;
b. separately passing the drug-resin granules and excipients except the sweetener, flavors, and lubricant, through a #45 sieve (ASTM);
c. separately passing the sweetener, flavors and extra-granular lubricant through a #60 sieve (ASTM);
d. blending the drug-resin granules with the diluent, coloring agent and disintegrants in a double cone blender for about 10 to 20 minutes;
e. blending the product of step (d) with the sieved sweeteners, flavors and lubricant in a double cone blender for about 4 to 8 minutes; and
f. compressing the blended ingredients into an ODT using direct compression.

For compression of the mouth dissolving tablets, the die-punch tool of 10.5 mm round shaped punch was used. In some embodiments, any other appropriate size or shape tool may be used. In some embodiments, the ODTs can be made using a dry granulation process. The prepared tablets exhibited hardness of about 3-12 kp with disintegration time of less than a minute, and preferably 10 to 30 seconds.

The following exemplary ODT formulations comprising Drotaverine were prepared by preparation methods illustrated herein above by employing varying amount of the drug-resin complex.

### Example 3: An Exemplary Formulation Comprising Drotaverine was Prepared by Employing Drug-Resin Complex

TABLE 1
Formulation comprising 63.60 mg Drotaverine-resin complex

| Ingredient | mg/tablet |
|---|---|
| Drotaverine | 21.20 |
| Kyron 314 | 42.40 |
| MCC pharmaceutical grade (PH) 102 | 42.76 |
| Mannitol 300 DC | 15.00 |
| Crospovidone | 7.00 |
| Aspartame | 0.50 |
| Lemon flavor | 2.00 |
| Magnesium stearate | 1.00 |

### Example 4: An Exemplary Formulation Comprising Drotaverine was Prepared by Employing Drug-Resin Complex

TABLE 2
Formulation comprising 106.00 mg Drotaverine-resin complex

| Ingredient | mg/tablet |
|---|---|
| Drotaverine | 21.20 |
| Kyron 114 | 84.80 |
| MCC PH 102 | 42.76 |
| Xylitol | 27.00 |
| Croscarmellose Sodium | 7.00 |
| Sodium Saccharin | 2.50 |
| Berry flavor | 2.00 |
| Magnesium stearate | 2.00 |

### Example 5: An Exemplary Formulation Comprising Drotaverine was Prepared by Employing Drug-Resin Complex

TABLE 3
Formulation comprising 84.80 mg Drotaverine-resin complex

| Ingredient | mg/tablet |
|---|---|
| Drotaverine | 21.20 |
| Indion 234 | 63.60 |
| MCC PH 102 | 42.76 |
| Sorbitol DC | 70.00 |
| Croscarmellose Sodium | 9.00 |
| Sodium Saccharin | 3.00 |
| Strawberry flavor | 4.00 |
| Magnesium stearate | 2.00 |

### Example 6: An Exemplary Formulation Comprising Drotaverine was Prepared by Employing Drug-Resin Complex

TABLE 4
Formulation comprising 212.00 mg Drotaverine-resin complex

| Ingredient | mg/tablet |
|---|---|
| Drotaverine | 42.40 |
| Indion 294 | 169.60 |
| MCC PH 102 | 74.00 |
| Sorbitol DC | 70.00 |
| Croscarmellose Sodium | 9.00 |
| Sodium Saccharin | 4.00 |
| Mango flavor | 4.00 |
| Sodium Stearyl Fumarate | 2.00 |

### Example 7: An Exemplary Formulation Comprising Drotaverine was Prepared by Employing Drug-Resin Complex

TABLE 5
Formulation comprising 75.40 mg Drotaverine-resin complex

| Ingredient | mg/tablet |
|---|---|
| Drotaverine | 10.60 |
| Indion 204 | 64.80 |
| MCC PH 102 | 42.76 |
| Sorbitol DC | 70.00 |
| Croscarmellose Sodium | 9.00 |
| Sucralose | 6.00 |
| Licorice powder | 3.50 |
| Raspberry flavor | 4.00 |
| Stearic acid | 2.00 |

### Example 8: An Exemplary Formulation Comprising Drotaverine was Prepared by Employing Drug-Resin Complex

TABLE 6
Formulation comprising 125.30 mg Drotaverine-resin complex.

| Ingredient | mg/tablet |
|---|---|
| Drotaverine | 40.50 |
| Kyron 114 | 84.80 |
| MCC PH 102 | 42.76 |
| Sorbitol DC | 70.00 |
| Croscarmellose Sodium | 9.00 |
| Sucralose | 6.00 |
| Tartrazine lake color | 1.50 |
| Orange flavor | 4.00 |
| Mint Flavor | 3.00 |
| Magnesium Stearate | 4.00 |

### Example 9: An Exemplary Formulation Comprising Drotaverine was Prepared by Employing Drug-Resin Complex

TABLE 7
Formulation comprising 160.50 mg Drotaverine-resin complex.

| Ingredient | mg/tablet |
|---|---|
| Drotaverine | 40.50 |
| Kyron 314 | 120.00 |
| MCC PH 102 | 42.76 |
| Pearlitol 200SD | 60.00 |
| Croscarmellose Sodium | 9.00 |
| Aspartame | 6.00 |
| Tartrazine lake color | 1.50 |
| Lemon flavor | 2.00 |
| Vanilla flavor | 3.00 |
| Bitter Masking flavor | 5.00 |
| Magnesium Stearate | 4.00 |

### Example 10: An Exemplary Formulation Comprising Drotaverine was Prepared by Employing Drug-Resin Complex

TABLE 8
Formulation comprising 141.50 mg Drotaverine-resin complex.

| Ingredient | mg/tablet |
|---|---|
| Drotaverine | 20.20 |
| Indion 234 | 121.3 |
| MCC PH 102 | 47 |
| Pearlitol 200SD | 160 |
| Croscarmellose Sodium | 20 |
| Aspartame | 16 |
| Tartrazine lake color | 1.5 |
| Lemon flavor | 2 |
| Vanilla flavor | 3 |
| Bitter Masking flavor | 5 |
| Magnesium Stearate | 4 |

### Example 11: An Exemplary Formulation Comprising Drotaverine was Prepared Drug-Resin Complex

TABLE 9
Formulation comprising 200.50 mg Drotaverine-resin complex.

| Ingredient | mg/tablet |
|---|---|
| Drotaverine | 40.50 |
| Kyron 114 | 160.00 |
| MCC PH 102 | 49.00 |
| Pearlitol 200SD | 60.00 |
| Croscarmellose Sodium | 9.00 |
| Aspartame | 6.00 |
| Tartrazine lake color | 1.50 |
| Tutti-fruity flavor | 2.00 |
| Vanilla flavor | 3.00 |
| Magnesium Stearate | 4.00 |

### Example 12: An Exemplary Formulation Comprising Drotaverine was Prepared by Employing Drug-Resin Complex

TABLE 10
Formulation comprising 81.20 mg Drotaverine-resin complex.

| Ingredient | mg/tablet |
|---|---|
| Drotaverine | 20.50 |
| Indion 204 | 60.70 |
| MCC PH 102 | 89.00 |
| Maltodextrin DC | 60.00 |
| Croscarmellose Sodium | 9.00 |
| Aspartame | 6.00 |
| Tartrazine lake color | 5.80 |
| Tutti-fruity flavor | 2.00 |
| Vanilla flavor | 3.00 |
| Magnesium Stearate | 4.00 |

### Example 13: An Exemplary Formulation Comprising Drotaverine was Prepared by Employing Drug-Resin Complex

TABLE 11
Formulation comprising 180.20 mg Drotaverine-resin complex.

| Ingredient | mg/tablet |
|---|---|
| Drotaverine | 20.20 |
| Indion 294 | 160.00 |
| Starch maize | 69.00 |
| Mannitol DC | 75.30 |
| Croscarmellose Sodium | 9.00 |
| Aspartame | 6.00 |
| Tartrazine lake color | 1.50 |
| Mango flavor | 2.00 |
| Vanilla flavor | 3.00 |
| Glyceryl Behenate | 4.00 |

### Example 14: An Exemplary Formulation Comprising Drotaverine was Prepared by Employing Drug-Resin Complex

TABLE 12
Formulation comprising 81.20 mg Drotaverine-resin complex.

| Ingredient | mg/tablet |
|---|---|
| Drotaverine | 20.50 |
| Kyron 114 | 60.70 |
| MCC PH 102 | 89.00 |
| Maltodextrin | 60.00 |
| Croscarmellose Sodium | 9.00 |
| Aspartame | 6.00 |
| Tartrazine lake color | 5.80 |
| Strawberry Flavor | 2.00 |
| Vanilla Flavor | 3.00 |
| Stearic acid | 4.00 |

### Example 15: An Exemplary Formulation Comprising Drotaverine was Prepared by Employing Drug-Resin Complex

TABLE 13
Formulation comprising 201.00 mg Drotaverine-resin complex.

| Ingredient | mg/tablet |
|---|---|
| Drotaverine | 41.00 |
| Kyron 114 | 160.00 |
| Prosolv ODT (JRS Pharma) | 80.00 |
| Crospovidone | 22.00 |
| Licorice powder | 8.00 |
| Aspartame | 12.00 |
| Tartrazine lake color | 5.80 |
| Peppermint flavor | 2.00 |
| Vanilla flavor | 3.00 |
| Stearic acid | 4.00 |

### Example 16: An Exemplary Formulation Comprising Drotaverine was Prepared by Employing of Drug-Resin Complex

TABLE 14
Formulation comprising 201.00 mg Drotaverine-resin complex.

| Ingredient | mg/tablet |
|---|---|
| Drotaverine | 41.00 |
| Kyron 114 | 160.00 |
| Pharmaburst (SPI Pharma) | 100.00 |
| Sodium Starch Glycollate | 24.00 |
| Sucralose | 12.00 |
| Tartrazine lake color | 5.80 |
| Peppermint flavor | 2.00 |
| Vanilla flavor | 3.00 |
| Stearic acid | 4.00 |

### Example 17: An Exemplary Formulation Comprising Drotaverine was Prepared by Employing Drug-Resin Complex

TABLE 15
Formulation comprising 320.00 mg Drotaverine-resin complex.

| Ingredient | mg/tablet |
|---|---|
| Drotaverine | 80.00 |
| Kyron 114 | 240.00 |
| Prosolv ODT (JRS Pharma) | 70.00 |
| Crospovidone | 22.00 |
| Licorice powder | 8.00 |
| Aspartame | 12.00 |
| Tartrazine lake color | 7.00 |
| Peppermint flavor | 2.00 |
| Raspberry flavor | 3.00 |
| Magnesium Stearate | 4.00 |

### Example 18: An Exemplary Formulation Comprising Drotaverine was Prepared by Employing Drug-Resin Complex

TABLE 16
Formulation comprising 14.50 mg Drotaverine-resin complex.

| Ingredient | mg/tablet |
|---|---|
| Drotaverine | 2.5 |
| Kyron 114 | 12.0 |
| MCC PH 102 | 15.00 |
| Sorbitol DC | 29.50 |
| Croscarmellose Sodium | 3.00 |
| Sucralose | 2.00 |
| Orange flavor | 4.00 |
| Calcium Stearate | 2.00 |

### Example 19: Drug Dissolution Study

The ODT prepared using the ingredients illustrated in tables 1 to 16 above were evaluated using an in vitro drug dissolution study as described below in Table 17 for a representative formulation.

The ODT formulation showed quick drug release in the mouth compared to the 40 and 80 mg film coated tablets. The developed tablets of present disclosure showed faster drug release. In view of the drug dissolution study described herein, the ODTs of the present disclosure should lead to faster pain relief than the currently available film coated tablets. The ODT formulation is expected to exhibit quick onset of action as almost 90% drug release is achieved in about 5 minutes as shown in Table 17 below.

TABLE 17
Comparative drug dissolution data for Drotaverine ODT of
present disclosure vs. commercially available Drotin ® film
coated tablets of 40 and 80 mg strengths
Comparative drug dissolution data for Film Coated vs.
mouth dissolving Tabs

| Dissolution Condition | |
|---|---|
| Apparatus | Paddle |
| RPM | 40 |
| Sample withdrawn | 10 |
| Media Volume | 750 |
| Dissolution Media | 0.1N HCl |

| | % Drug Dissolved | | |
|---|---|---|---|
| | Drotaverine | Drotaverine | Drotaverine |
| | 40 ODT | 40 mg | 80 mg |
| Time (in | (Present | (Drotin ® Film | (Drotin ® Film |
| minutes) | disclosure) | coated Tabs) | coated Tabs) |
| 05 | 85-90 | 27 | 14 |
| 15 | 90-95 | 59 | 41 |
| 60 | 97-100 | 75 | 65 |

### Example 20: Study of Tablet Hardness and Disintegrating Property of ODTs of Present Disclosure

The ODTs prepared using the steps described in Examples 1 and 2 and using the ingredients described in Tables 1-16 above exhibited hardness of about 3-12 kp with disintegration time of less than a minute when placed in 1-3 mL of water. For a tablet with a 3-5 kp hardness, the disintegration time was about 8-12 seconds. For a tablet with a 10-12 kp hardness, the disintegration time was about 25-30 sections.

### Example 20: Stability Data

The ODT formulations developed as per the present disclosure demonstrated chemical and physical stability using ICH accelerated stability guidelines (40° C.±2° C. and 75% RH±5% RH). The accelerated stability data of a Drotaverine tablet formulated without a drug-resin complex is presented in Table 18 below, while the accelerated stability data of an ODT using a drug-resin complex according to Example 16 is presented below in Table 19 below:

TABLE 18
Tablet without Drug-resin resin complex

| | | Cream yellow | Cream yellow | light yellow | light yellow | light yellow |
|---|---|---|---|---|---|---|
| | | colour, round, | colour, round, | colour, round, | colour, round, | colour, round, |
| | | biconvex, | biconvex, | biconvex, | biconvex, | biconvex, |
| | | uncoated | uncoated | uncoated | uncoated | uncoated |
| Description | | tablets | tablets | tablets | tablets | tablets. |
| Assay | | 39.3 mg | 38.7 mg | 38.1 mg | 37.6 mg | 37.2 mg |
| Water by KF | | 3.76% | 4.21% | 5.23% | 5.60% | 6.50% |
| Related | Drotaverladine | 0.92 | 1.7 | 2.3 | 2.8 | 3.2 |
| Substance | Highest | 0.12 | 0.51 | 0.68 | 0.82 | 0.91 |
| | Unknown | | | | | |
| | Total | 1.09 | 2.35 | 3.72 | 4.43 | 5.89 |
| | Impurities | | | | | |

TABLE 19
ODT with drug-resin complex according to Example 16

| | | Cream yellow | Cream yellow | Cream yellow | Cream yellow | Cream yellow |
|---|---|---|---|---|---|---|
| | | colour, round, | colour, round, | colour, round, | colour, round, | colour, round, |
| | | biconvex, | biconvex, | biconvex, | biconvex, | biconvex, |
| | | uncoated | uncoated | uncoated | uncoated | uncoated |
| Description | | tablets | tablets | tablets | tablets | tablets. |
| Assay | | 40.70 mg | 40.74 mg | 40.32 mg | 39.91 mg | 39.83 mg |
| Water by KF | | 4.09% | 4.80% | 5.10% | 5.20% | 6.10% |
| Related | Drotaverladine | 0.15 | 0.16 | 0.22 | 0.20 | 0.21 |
| Substance | Highest | 0.08 | 0.07 | 0.13 | 0.16 | 0.17 |
| | Unknown | | | | | |
| | Total | 0.31 | 0.30 | 0.42 | 0.63 | 0.72 |
| | Impurities | | | | | |

The surprising, novel formulations and methods for preparation thereof as set forth in the present disclosure accurately describe the efficacy and utility of these formulations and methods to restore healthy functioning in humans and treat the conditions and disorders in humans as identified and described in this patent application.

Although the subject matter has been described herein with reference to certain preferred embodiments thereof, other embodiments are possible. For illustrative purpose, the formulations of the present disclosure comprise Drotaverine or salt thereof as the anti-spasmodic agent. However, those skilled in the art would appreciate that scope of the disclosure extends to formulations comprising other anti-spasmodic agents known in the field of art

1. An oral dosage form comprising Drotaverine or a salt thereof and an ion exchange resin, wherein Drotaverine and the ion-exchange resin comprise a Drotaverine-ion-exchange complex, and wherein the dosage form is formulated as an orally disintegrating tablet.

2. The oral dosage form of claim 1, wherein the dosage form further comprises a taste enhancer.

3. The oral dosage form of claim 2, wherein the dosage form further comprises a bitter masking flavor.

4. The oral dosage form of claim 1, wherein the dosage form comprises 2.5-100 mg Drotaverine or a salt thereof.

5. The oral dosage form of claim 1, wherein the dosage form comprises Drotaverine hydrochloride.

6. (canceled)

7. The oral dosage form of claim 1, wherein the ion-exchange resin is Kyron 114, Kyron 314, Indion 204, Indion 234, Indion 294, or a combination thereof.

8. The oral dosage form of claim 1, wherein the ratio of Drotaverine to ion-exchange resin is 1:2 to 1:6.

9. The oral dosage form of claim 1, wherein the oral dosage form is in the form of granules, wherein the size of the granules is in the range of around 850 microns to 300 microns.

10. The oral dosage form of claim 1, wherein the dosage form has a hardness of 3-12 kp.

11. The oral dosage form of claim 1, wherein the dosage form disintegrates in the mouth in 60 seconds or less without water.

12. (canceled)

13. The oral dosage form of claim 1, wherein the dosage form disintegrates in 1-3 mL of water in 60 seconds or less.

14. (canceled)

15. The oral dosage form of claim 1, wherein the dosage form further comprises excipients selected from a diluent, a disintegrant, a flavoring agent, a sweetener, a lubricant, a glidant, an anti-adherent, or a combination thereof.

16. The oral dosage form of claim 1, wherein the ratio of excipients to drug-resin complex is from 0.05:1 to 2.5:1.

17. The oral dosage form of claim 16, wherein the ratio of excipients to drug-resin complex is from 0.1:1 to 2:1.

18. The oral dosage form of claim 1, wherein the dosage form further comprises at least one diluent, wherein the diluent comprises a monosaccharide, an oligosaccharide, a polysaccharide, a lactose, a sugar alcohol, a cellulose powder, a microcrystalline cellulose, a silicified microcrystalline cellulose, a derivative of cellulose modified chemically, a starch, or a combination thereof.

19. The oral dosage form of claim 1, wherein the dosage form further comprises at least one disintegrant, wherein the disintegrant comprises at least one of a starch, natural or chemically modified cellulose, microcrystalline cellulose, gum, alginic acid or salt thereof, sugar, aluminum oxide, or a synthetic polymer.

20. The oral dosage form of claim 19, wherein the level of disintegrant was in the amount of 1% to 10% w/w.

21. (canceled)

22. (canceled)

23. (canceled)

24. (canceled)

25. The oral dosage form of claim 1, wherein the dosage form further comprises at least one flavoring agent, wherein the flavoring agent comprises a strawberry flavor, a cherry flavor, an orange flavor, a peppermint flavor, a black currant flavor, a banana flavor, a raspberry flavor, a red fruit flavor, a wild berry flavor, a caramel flavor, or a combination thereof.

26. The oral dosage form of claim 1, wherein the dosage form further comprises at least one sweetener, wherein the sweetener comprises aspartame, potassium acesulfame, sodium saccharinate, neohesperidine dihydrochalcone, sucralose, sucrose, fructose, monoammonium glycyrrhizinate, or a combination thereof.

27. The oral dosage form of claim 1, wherein the dosage form further comprises at least one lubricant, wherein the lubricant comprises sodium benzoate, sodium stearyl fumarate, calcium stearate, magnesium stearate, zinc stearate, glyceryl behenate, stearic acid, glyceryl monostearate, or a combination thereof.

28. An oral dosage form comprising Drotaverine or a salt thereof, Kyron 114, microcrystalline cellulose, colloidal silicon dioxide, mannitol, fructose, sucralose, aspartame, crospovidone, sodium stearyl fumarate, fruit flavoring, and bitter masking flavoring, wherein the dosage form is formulated as an orally disintegrating tablet.

29. (canceled)

30. The oral dosage form of claim 1, wherein the dosage form is stable for six months when stored at 40° C. and 75% RH as accelerated storage conditions.

31. (canceled)

32. (canceled)

33. The oral dosage form of claim 1, wherein the dosage form is stable for two years when stored at 25-30° C. temperature and 60-75% RH as long-term storage conditions.

34. The oral dosage form of claim 1, wherein the oral dosage form exhibits improved taste.

35. The oral dosage form of claim 1, wherein the oral dosage form exhibits improved aftertaste.

36. The oral dosage form of claim 1, wherein the oral dosage form exhibits improved palatability.

37. A method of preparing an orally disintegrating tablet of Drotaverine or salt thereof comprising:
a. mixing an ion-exchange resin with water;
b. adding Drotaverine or salt thereof to the ion-exchange resin dispersion of Step-a mixture to form a drug-resin complex by stirring for sufficient time;
c. washing the filtered drug-resin complex with water until the filtrate obtained after washing until unbound Drotaverine drug content is less than 2.0%;
d. drying the drug-resin complex to form a dried drug-resin complex which should contain less than 5.0% w/w water content;
e. milling and sieving the dried drug-resin complex to obtain drug-resin complex granules of appropriate size;
f. separately weighing the drug-resin complex granules and at least one excipient;
g. passing the drug-resin complex granules and excipient through a mesh;
h. blending the drug-resin complex granules and excipient; and subsequently mixed with disintegrant, flavoring agent, sweetener, lubricant, glidant, an anti-adherent; and
i. compressing the final blended drug-resin complex granules and excipients into as orally disintegrating tablets.

38. (canceled)

39. (canceled)

40. (canceled)

41. (canceled)

42. (canceled)

43. (canceled)

44. (canceled)

45. The method of claim 37, wherein the orally disintegrating tablet is formed using direct compression, dry granulation, wet granulation, or fluidized bed preparation methods.

46. The dosage form of claim 1, wherein at least 90% of the orally disintegrating tablet is dissolved in five minutes in vitro using a paddle at 40 RPM in 0.1 N HCl dissolution media.

47. A method of treating abdominal pain comprising administering to a subject in need thereof an oral dosage form comprising Drotaverine or a salt thereof, wherein the oral dosage form comprises the dosage form of claim 1.

48. (canceled)
